(19) United States
(12) Patent Application Publication
(10) Pub. No.: US 2008/0027422 A1
(43) Pub. Date: Jan. 31, 2008

(54) Closed-Loop Cryosurgical System and Cryoprobe

(75) Inventors: David W. Vancelette, San Diego, CA (US); Douglas A. Devens, Highland Park, IL (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/828,153

(22) Filed: Jul. 25, 2007

Related U.S. Application Data
(60) Provisional application No. 60/820,290, filed on Jul. 25, 2006.

(51) Int. Cl. A61B 18/02

(52) U.S. Cl. 606/23; 606/20; 62/51.2

(57) ABSTRACT

A closed loop cryosurgical system utilizing a plurality of cryoprobes to perform a cryosurgical treatment. The cryoprobes individually connect to a manifold portion of the cryosurgical system with a quick-connect coupling. Each cryoprobe includes a flexible conduit portion that is vacuum insulated leaving a freeze length of a probe end portion exposed on which ice ball formation may occur. The insulation space may be evacuated through an insulation channel by one or more of a getter chamber located within the cryostat, a vacuum pump located within the console, or by activation of one of the console compressors to pull the gases out of the insulation space prior to introduction of refrigerant into the circuit. The fluid pathways through which refrigerant travels through the console, cryostat, couplers, and cryoprobes can also be evacuated prior to a cryosurgical procedure.

### PRIORITY CLAIM

The present application claims priority to U.S. Provisional Application No. 60/820,290, filed Jul. 25, 2006, and entitled “CLOSED LOOP CRYOSURGICAL SYSTEM AND CRYOPROBE”, which is herein incorporated by reference in its entirety.

### FIELD OF THE INVENTION

This invention relates to cryosurgical probes for use in the treatment of cancerous tumors or lesions and more particularly to a closed loop cryosurgical system having multiple probes.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including breast cancer, liver cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

### SUMMARY OF THE INVENTION

The present disclosure is directed to a closed loop cryosurgical system having multiple probes. A closed loop cryosurgical system includes a console having a primary compressor for pressurizing a primary refrigerant, a secondary compressor for pressurizing a secondary refrigerant, and controls for controlling system parameters. A display is attached to the console so that an operator can monitor the system. High pressure refrigerant flows from the console to a cryostat heat exchanger module through a flexible refrigerant line. The cryostat includes a pre-cool heat exchanger (“pre-cooler”) and a recuperative heat exchanger (“recuperator”). High pressure secondary refrigerant is expanded and cools the high pressure primary refrigerant in the pre-cooler, then returns back to the console to be repressurized. The high pressure primary refrigerant is further cooled in the recuperator by the low pressure, low temperature primary refrigerant on its way back to the console. The high pressure primary refrigerant then passes into a plurality of flexible probes coupled to a manifold portion of the cryostat where it is expanded to further lower the temperature sufficient to allow ice ball formation and selective freezing of diseased tissue before it returns back to the console to be repressurized.

The present disclosure is also directed towards a plurality of flexible probes used in a closed loop cryosurgical system. Each probe connects to the manifold portion of the cryostat with a quick-disconnect coupling having fluid communication channels for the low pressure and high pressure primary refrigerant. A flexible conduit portion of each probe is vacuum insulated leaving a freeze length of a probe end portion exposed on which ice ball formation may occur. The insulation space may be evacuated by one or more of a getter chamber located within the cryostat, a vacuum pump located within the console, or by activation of one of the console compressors to pull the gases out of the insulation space prior to introduction of refrigerant into the circuit.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a view of an embodiment of a closed loop cryosurgical system according to the present disclosure.

FIG. 2 is a view of an embodiment of a cryostat heat exchanger module according to the present disclosure.

FIG. 3 is a view of an embodiment of a portion of a closed loop cryosurgical system according to the present disclosure.

### DETAILED DESCRIPTION OF THE INVENTION

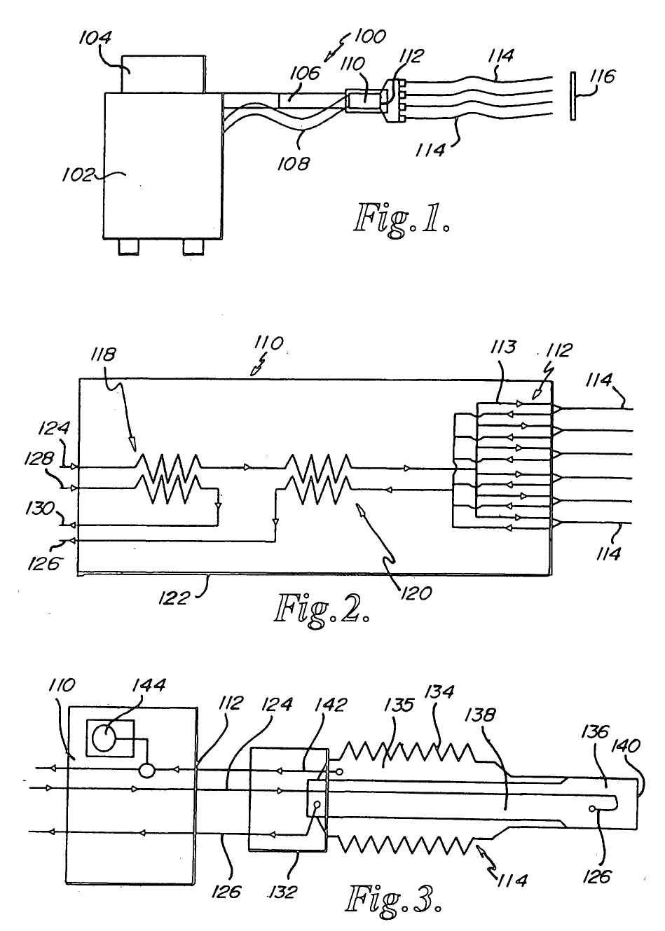

Referring to FIG. 1, there can be seen an embodiment of a closed loop cryosurgical system 100 according to the present disclosure. Cryosurgical system 100 includes a refrigeration and control console 102 with an attached display 104. Console 102 contains a primary compressor which provides a primary pressurized, mixed gas refrigerant to the system and a secondary compressor that provides a secondary pressurized, mixed gas refrigerant to the system. Adequate gas mixtures are known in the art that provide a dramatic increase in cooling performance over a single gas. Console 102 also contains controls that allow activation, deactivation, and modification of various system parameters, such as the flow rates, pressures, and temperatures of the refrigerants. Display 104 allows the operator to monitor, and in some embodiments adjust the system to ensure it is performing properly and can provide continuous historical and instantaneous display and recording of system parameters. An exemplary console that may be used with an embodiment of the present invention is used as part of the Her Option® Office Cryoablation Therapy available from American Medical Systems of Minnetonka, Minn.

The high pressure primary refrigerant is transferred to a cryostat heat exchanger module 110 through a flexible line 108. As illustrated in FIG. 2, cryostat 110 includes a manifold portion 112 having a plurality of pathways 113 that transfer the refrigerant into and receive refrigerant out of a plurality of flexible probes 114. The cryostat 110 and flexible probes 114 are also connected to the console by way of an articulating arm 106, which may be manually or automatically used to position the cryostat 110 and flexible probes 114. Although depicted as having the flexible line 108 separate from the articulating arm 106, cryosurgical system 100 may incorporate the flexible line 108 within the articulating arm 106. A positioning grid 116 may be used to properly align and position the flexible probes 114 for patient insertion.

Referring again to FIG. 2, cryostat 110 comprises both a pre-cool heat exchanger, or pre-cooler 118, and a recuperative heat exchanger, or recuperator 120. A vacuum insulated jacket 122 surrounds the cryostat 110 to prevent the ambient air from warming the refrigerant within the cryostat 110 and to prevent the outer surface of the cryostat 110 from becoming excessively cold. High pressure primary refrigerant 124 enters the cryostat 110 and is cooled by high pressure secondary refrigerant 128 that is expanded to a lower temperature in the pre-cool heat exchanger 118. The resulting low pressure secondary refrigerant 130 then returns to the secondary compressor to be repressurized. Since the secondary refrigerant does not flow into the probes 114 (which are brought into direct contact with the patient), a higher pressure can be safely used for the secondary refrigerant 128, 130 than the primary refrigerant 124, 126.

The high pressure primary refrigerant 124 then continues into the recuperator 120 where it is further cooled by the low pressure primary refrigerant 126 returning from the manifold 112. The low pressure primary refrigerant 126 is colder than the high pressure primary refrigerant because it has undergone Joule-Thompson expansion in the plurality of probes 114. Recuperator 120 is preferably incorporated into the cryostat 110. Alternatively, tubing coils inside each probe 114 may act as recuperative heat exchangers in order to reduce insulation requirements and return low pressure refrigerant to the console.

After leaving the recuperator, high pressure primary refrigerant 124 flows into the manifold 112, where it is distributed into multiple flexible probes 114. In one representative embodiments, six flexible probes 114 are connected to the manifold, but one of skill in the art will recognize that greater or fewer probes may be used depending on the needs of a particular procedure. In each flexible probe 114, the refrigerant 124 flows into a Joule-Thompson expansion element, such as a valve, orifice, or other type of flow constriction, located near the tip of each flexible probe 114, where the refrigerant 124 is expanded isenthalpically to a lower temperature. In one presently preferred embodiment, the Joule-Thompson expansion elements are capillary tubes. The refrigerant then cools a heat transfer element mounted in the wall of the probe, allowing the probe to form ice balls that freeze diseased tissue. The expanded refrigerant then takes the low pressure primary refrigerant path 126, exits the manifold 112, travels through the recuperator 120 (where it serves to further cool the high pressure primary refrigerant 124), flows past the precooler 118 and back to the primary compressor in the console, where it is compressed back into high pressure refrigerant 124 so that the above process can be repeated.

Referring now to FIG. 3, there can be seen an embodiment of a vacuum insulated flexible probe 114 according to the present disclosure. Probe 114 includes a quick-disconnect coupling 132 that mates with the manifold portion 112 of the cryostat 110 to connect the probe 114 to the system. The quick-disconnect coupling 132 includes pathways for high pressure primary refrigerant 124 and low pressure primary refrigerant 126 to flow between the cryostat 112 and the probe 114. Probe 114 also includes a flexible conduit 134 and a rigid probe end 138 contained partially therein. Flexible conduit 134 may incorporate bellows, corrugate, convoluted or Nitinol tubing to increase flexibility. Flexible conduit 134 covers only a portion of rigid probe end 138, leaving freeze portion 136 exposed. Freeze portion 136 is preferably 30-40 mm long and the tip 140 of freeze portion 136 is preferably about 2.1 mm in diameter. Probe 114 further includes fluid pathways 124, 126 for high pressure primary refrigerant and low pressure primary refrigerant. Probe 114 also includes a Joule-Thompson expansion element to expand (and thereby further cool) the high pressure primary refrigerant 124.

As illustrated in FIG. 3, quick-disconnect coupling 132 may be located at the proximal end of probe 114 at its connection with manifold 112. In this configuration, the entire probe 114 is disposable. Alternatively, the vacuum insulated flexible conduit 134 may be permanently attached to manifold 112, with the quick-disconnect coupling connected to the proximal end of the rigid probe end 138 such that only rigid probe end 138 is disposable.

In order to actively evacuate an insulation space 135 within the flexible conduit 134, an additional insulation communication channel 142 through the quick-disconnect coupling 132 between the probe 114 and the cryostat 110 is required. Because of this, each time the quick-disconnect coupling 132 is connected to the cryostat 110, air will be introduced into the insulation space 135. Air can be evacuated from the insulation space 135, however, by one or more of a getter chamber 144 located within the cryostat, a vacuum pump located within the control console 102, or by activation of one of the console compressors to pull the gases out of the insulation space prior to introduction of refrigerant into the circuit. For example, prior to operation of the system a compressor in the console and/or other vacuum pumps may be used to evacuate gases not only from the insulation space 135 through the insulation communication channel 142, but also from the high pressure primary refrigerant 124 and low pressure primary refrigerant 126 channels. The probe 114 can then be connected to a pre-activated getter chamber 144 in the cryostat 110 held at a low pressure to maintain the required low vacuum in the insulation space 135 while the system is in operation.

Maintaining a vacuum within insulation space 135 surrounding portions of the probe serves multiple functions. It limits the freeze portion 136 which makes it easier to confine the freezing process to a small area of damaged tissue. It also helps maintain the low temperature of the low pressure primary refrigerant 126 as it returns to the cryostat. This allows the low pressure primary refrigerant 126 to better cool the high pressure primary refrigerant 124 in the recuperator 120. It also helps prevent unwanted frosting and low temperatures on the outer jacket of the flexible conduit 134. In addition, creating a vacuum within the insulation space 135 with control console 102 based pumping and/or getter evacuation also reduces the cost and complexity of manufacturing the probes 114. Alternatively, foam, aerogel, air, or noble gas gaps can also be used for insulation.

The disclosed closed loop cryosurgical system with multiple probes provides a system that is compact, mobile and reliable. The system further eliminates the need for gas replenishment or cylinder replacement, which reduces the cost and maintenance of the system relative to open loop systems. A closed loop cryosurgical system according to the present disclosure may be used to treat cancerous tumors or lesions in the prostate, kidneys or other organs/tissue.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A closed loop cryosurgical system comprising:
a console having a primary compressor for pressurizing a high pressure primary refrigerant and a secondary compressor for pressurizing a secondary high pressure refrigerant;
a cryostat heat exchanger having a precooler, a recuperator and a probe manifold, and
a plurality of cryoprobes individually, fluidly connected to the probe manifold with a quick-connect coupling, each cryoprobe having a flexible conduit partially covering a rigid probe end so as to define an exposed freeze portion and an insulation space, and wherein an insulation channel operably couples the insulation space to the quick-connect coupling such that upon connection of the quick-connect coupling to the probe manifold, any air within the insulation space can be evacuated to form a vacuum in the insulation space.

2. The closed loop cryosurgical system of claim 1, wherein the cryostat heat exchanger further includes a getter chamber fluidly coupled to the probe manifold and wherein the getter chamber evacuates and maintains the vacuum within the insulation space.

3. The closed loop cryosurgical system of claim 1, wherein the console further includes a vacuum pump wherein the vacuum pump evacuates the insulation space through the insulation channel to form the vacuum within the insulation space.

4. The closed loop cryosurgical system of claim 1, wherein the primary compressor evacuates the insulation space through the insulation channel to form the vacuum within the insulation space.

5. The system of claim 3 or 4, wherein the secondary compressor evacuates the insulation space through the insulation channel to form the vacuum within the insulation space.

6. The system of claim 1, wherein the quick-connect coupling allows each cryoprobe to be individually attached to and detached from the probe manifold.

7. A cryoprobe for use in a closed loop cryosurgical system, comprising:
a probe body having a refrigerant flow circuit including a high pressure fluid supply pathway, an expansion element and a low pressure fluid return pathway;
a flexible conduit partially surrounding the probe body to define an insulation space and a freeze portion at a rigid probe end; the insulation space fluidly connected to an insulation channel,
a coupling adapted to connect the probe body to a cryosurgical system, the coupling having a refrigerant supply connection, a refrigerant return connection and an insulation channel connection,
wherein air within the insulation space can be evacuated through the insulation channel connection to form a vacuum within the insulation space.

8. The cryoprobe of claim 7, wherein the expansion element comprises a Joule-Thompson expansion element selected from the group consisting of: a valve, an orifice and a capillary tube.

9. The cryoprobe of claim 7, wherein the freeze portion has a conductive freeze length of about 30 mm to about 40 mm.

12. The cryoprobe of claim 7, wherein the freeze length is between 30 and 40 mm.

13. The cryoprobe of claim 7, wherein the rigid probe end had a tip diameter of about 2.1 mm.

14. The cryoprobe of claim 7, wherein the flexible conduit is selected from the group consisting of: bellows tubing, corrugated tubing, convoluted tubing and nitinol tubing.

15. A method for improving cooling performance of a cryoprobe, comprising:
connecting a plurality of cryoprobes to a manifold portion of a cryosurgical system with a quick-connect coupling, the quick-connect coupling connecting refrigerant supply lines, refrigerant return lines and an insulation channel between each cryoprobe and the cryosurgical system; and
evacuating air from an insulation space in each cryoprobe through the insulation channels to form a vacuum within the insulation space.

16. The method of claim 15, further comprising:
fabricating each cryoprobe such that a flexible conduit portion at least partially covers a probe body so as to define a freeze portion having a desired freeze length at a rigid probe end.

17. The method of claim 15, further comprising:
providing a cryostat heat exchanger having a getter chamber fluidly coupled to the manifold portion and wherein the getter chamber evacuates and maintains the vacuum within the insulation space.

18. The method of claim 15, further comprising:
providing a console having a primary compressor and a secondary compressor and wherein either the primary compressor or the secondary compressor evacuates the insulation space to form the vacuum.
